(19) United States
(12) Patent Application Publication
(10) Pub. No.: US 2015/0225749 A1
(43) Pub. Date: Aug. 13, 2015

(54) HIGH MOISTURE, RENEWABLE FEEDSTOCK USE IN INTEGRATED ANAEROBIC TREATMENT AND SYNGAS FERMENTATION TO ALCOHOLS

(71) Applicant: Robert Hickey, Okemos, MI (US)

(72) Inventor: Robert Hickey, Okemos, MI (US)

(21) Appl. No.: 14/176,013

(22) Filed: Feb. 7, 2014

(51) Int. Cl. C12P 7/14

(52) U.S. Cl. CPC C12P 7/14

(57) ABSTRACT

The present invention relates to use of high moisture, renewable feedstocks in integrated anaerobic digestion treatment (AD) and Syngas fermentation to alcohols and other soluble products. More specifically, the invention relates to use of high moisture, renewable feedstocks such as the organic fraction of municipal solid waste (OFMSW), biological waste sludge, source separated organics, green wastes such as FW from supermarkets, cafeterias, etc., and other such plentiful sources for power and fermentation, significantly increasing yield of a desired alcohol.

Even for relatively large Syngas to ethanol (or other chemical) production facilities, such wastes could represent a significant fraction of needed resources for Syngas fermentation. The proper integration of the anaerobic digestion process, post treatment, and use of fractionated components are also believed to represent a considerable savings in overall production costs.

### FIELD OF THE INVENTION

The present invention relates to use of, renewable feedstocks in integrated anaerobic digestion treatment (AD) and synthesis gas (Syngas) fermentation to alcohols and other soluble products. More specifically, the invention relates to the generation of a process fuel and a fermentation substrate at increased product yield and reduced cost by use of high moisture, renewable feedstocks such as the organic fraction of municipal solid waste (OFMSW); biological waste sludge; source separated organics; green wastes such as food wastes (FW) from supermarkets, cafeterias, etc.; and other such plentiful, renewable sources such as certain agricultural wastes and even specifically harvested “energy” crops.

### BACKGROUND

Certain waste materials such as the organic fraction of municipal solid waste [OFMSW], biological waste sludge, source separated organics, or green wastes (i.e. FW from supermarkets, cafeterias, etc.), high strength food processing wastewaters (whey, whey permeate, vegetable preparation WW, etc.), distillery byproducts including stillage and thin stillage and others although abundant can be problematic to use as feedstocks for gasification and subsequent fermentation to ethanol (and other liquid products). This is due to their high moisture content and/or the presence of compounds in the waste that make them hard to manage or produce undesirable compounds in the product Syngas. These wastes can be valuable sources of nutrients (such as nitrogen, sulfur and phosphorus), as well as CH₄ and CO₂, all useful for fermentation of Syngas to chemicals processes.

Even for relatively large Syngas to Alcohol (or other chemicals) production facilities these wastes can still represent a significant fraction of these needed resources for the Syngas fermentation process. The proper integration of the anaerobic processing, post treatment and use of the fractionated components can represent a considerable savings in overall production costs and result in an overall process that is much more sustainable.

Heretofore, the use of high moisture, renewable feedstocks in such area has been frowned upon due to due to their high moisture content and/or the presence of compounds in the waste that make them difficult to manage or which produce undesirable compounds in the Syngas feedstream. The instant invention overcomes these issues and secures even greater value from these materials

### BRIEF DESCRIPTION OF THE INVENTION

The present invention integrates the anaerobic digestion (AD) of high moisture organic feedstocks such as excess biosolids from a syngas fermenter operation, and additional supplemental outside sources of readily digestible renewable organics substrates to produce a biogas stream. This biogas is then reformed to syngas and blended with the production of a syngas from reforming natural gas (NG) at a resulting mixture that has the e⁻/C ratio necessary to allow a high conversion efficiency of both H₂/CO₂ and CO to soluble products in the syngas fermentation process.

The AD process also produces additional fermentation products and water that can be used in the syngas to chemicals fermentation process to potentially supply not only syngas, but also nitrogen (N), phosphorus (P) and H₂S, major nutrients required in the fermentation. Also in one embodiment recovery and reuse of valuable trace metals can be achieved.

The present invention discloses methods and systems for managing high moisture organic wastes and feedstocks as described herein, that simplifies the their treatment and facilitates their utilization to provide valuable sources of nutrients such as nitrogen, sulfur and phosphorus, as well as CH₄ and CO₂, which are all useful for fermentation of Syngas to soluble oxygenated products.

In one embodiment, the biogas can be reformed to syngas using a non-catalytic partial oxidation system and the syngas produced blended with syngas from another source to obtain the desired syngas composition to maximize utilization of all the carbon (CH₄, CO and CO₂) in the combined syngas feed to the liquid products generated in the syngas fermentation system. The partial reforming technology can include but is not limited to oxygen fed system of patent application or a plasma arc type gasification process. At least some of the H₂S produced in the AD is conserved in the syngas produced from the biogas and can be used as sulfur source in the syngas fermentation.

In another board aspect of the invention, nitrogen and phosphorus are converted to simple inorganic forms (NH₄ and PO₄) in the AD process and are recovered in a form that can be readily used back in the syngas fermentation.

In another embodiment, the digestate produced in the AD can be dewatered, dried, and gasified to produce an ash that contains essentially all of the minerals and trace metals required for the syngas fermentation. This ash can be processed to recover the metals in a form that can be reused in the syngas fermentation process making the overall system more sustainable.

The digestate from an Anaerobic Digester (AD), that receives the high moisture organic feedstocks, can potentially provide a source of water for use within or outside of the method operations.

Some of these waste streams and renewable feedstocks may require some form of pretreatment before they can be anaerobically digested (such as removal of contaminating materials, size reduction, thermal pretreatment, etc.,) but many of these wastes and organic streams can be directly treated anaerobically in methanogenic systems to produce a biogas containing primarily CH₄ and CO₂ along with some H₂S. The digestion process also converts the nitrogen and phosphorus in the high moisture organic feedstocks, that are often present in forms that not readily bioavailable to the microorganisms of the fermentation process (such as proteins and organic phosphorus containing compounds), into inorganic forms, such NH₄⁺/NH₃ and ortho PO₄ that are recoverable and bioavailable in the Syngas fermentation process. To prevent/reduce the potential for introducing biological contaminants, heat treatment (pasteurization or sterilization) or physical disruption methods can be employed as pretreatments or post-treatments for the recovered components being reused back in the fermentation.

### DEFINITIONS

Liquid products means Alcohol, acids such as acetic acid, propionic acid and butyric acid.

Alcohol means one or more alkanols containing two to six carbon atoms. In some instances alcohol is a mixture of alkanols produced by the microorganisms contained in the fermentation broth. The most common alcohols produced by this method are ethanol and butanol.

Electron to carbon ratio is calculated as the quotient of the quantity of two times the sum of the molar concentrations of carbon monoxide and hydrogen divided by quantity of the sum of the molar concentrations of carbon monoxide and carbon dioxide:

e⁻/C=2([CO]+[H2])/([CO]+[CO2]). Fermentation broth means a liquid water phase which may contain dissolved compounds including, but not limited to hydrogen, carbon monoxide, and carbon dioxide and required nutrients including N, P, and S, dissolved salts, and trace metals required for the optimal growth of the syngas fermenting organisms.

A concentration of Liquid Products, (primarily alcohol) below that which unduly adversely affects the rate of growth of the culture of microorganisms will depend upon the type of microorganism and the alcohol. An unduly adverse effect on the growth rate means that a significant, usually at least a 20 percent, decrease in the growth rate of the microorganisms is observed in comparison to the growth rate observed in an fermentation broth having about 10 grams per liter alcohol therein, all other parameters being substantially the same.

The term bubble column bioreactor as used herein refers to a deep tank bioreactor in which the syngas is introduced as a gas/liquid dispersion. It may be a conventional bubble column reactor loop type reactor or hybrids of these two generic type reactor processes unless otherwise explicitly stated. A commercial scale bioreactor has a capacity for fermentation broth of at least 1 million, and more preferably at least about 5 and more preferably in the range of 5 to 25 million, liters.

Renewable Wastes or High Moisture Renewable Organic Feedstocks means waste materials and other organic streams comprising organic matter derived from natural resources that provide a source of carbon that can be replaced in less than a millennium and in most cases in several years or less, as opposed to carbon sources that take at least a geologic age to replace once depleted. Examples comprise the organic fraction of municipal solid waste [OFMSW], biological waste sludge [biosolids], source separated organics, or green wastes (i.e. FW from supermarkets, cafeterias, etc.), high strength food processing wastewaters (whey, whey permeate, vegetable preparation wastes, food and beverage production wastewaters, etc.), distillery wastes and byproducts including stillage and thin stillage, certain agricultural wastes and specifically harvested “energy” crops.

Syngas means a gas containing at least one of hydrogen and carbon monoxide and may, and usually does, contain carbon dioxide and lesser amounts of H₂S, N₂, and CH₄.

### BRIEF DESCRIPTION OF THE DRAWING

FIG. 1 provides a generic, schematic diagram of one embodiment of the proposed system associated with the use of the methodology presented herein below.

FIG. 2 provides a generic, schematic diagram of a further embodiment of the proposed system associated with the use of the methodology presented herein below

FIG. 3 provides a generic, schematic diagram with a yet different embodiment of the proposed system associated with the use of the methodology presented herein below.

### SUMMARY OF THE INVENTION

To achieve the maximum value from use of high moisture renewable materials they must first be processed in an anaerobic treatment system to produce useable compounds which must next be segregated, and then subsequently integrated into the Syngas fermentation system. Some such waste streams may also require some form of pretreatment before they can be anaerobically digested, such as removal of contaminating materials, size reduction, etc. By this invention it has been found that many such waste streams can be treated anaerobically in methanogenic systems without pretreatment to produce a biogas containing primarily CH₄ and CO₂, typically in amounts of from 50 to 75% and 25 to 50%, respectively, as well as H₂S in a quantity of up to 3%. The digestion process also converts nitrogen and phosphorus in the waste streams into forms which are recoverable and useable in the Syngas fermentation process. Further processing of the digestate can be used to recover valuable trace metals and minerals for reuse.

Even for relatively large production facilities for the conversion of Syngas to oxygenated liquid products such as alcohols, such wastes could represent a significant fraction of the feed gas and nutrients needed for Syngas fermentation. The proper integration of anaerobic processing, post-treatment, and use of fractionated components will represent a considerable savings in overall production costs as well as a process that is much more sustainable. In addition, essentially all the carbon in the biogas is fixed as organic carbon products with the instant invention

By combining the biogas (directly or after reforming via a non-catalytic partial oxidation process) with a syngas from reforming natural gas (or other H₂ rich gas that has a high e⁻/C ratio) there exists the opportunity to fix the CO₂ in the biogas as a liquid product. This is done by combining the gases in a ratio to achieve a combined gas e⁻/C of between the optimal range of 6.4 to 5.8.

The remaining digestate from the anaerobic digestion of the high moisture renewable material contains a substantial amount of carbon as solids. Several methods are available for recovery of carbon from this digestate. The preferred method of this invention recovers this carbon through gasification of the solids to produce additional syngas. This option becomes economically viable if there is sufficient waste heat available from the syngas fermentation process to provide much of the drying energy requirements. In this manner the process of the invention will recover essentially of the available carbon from the high moisture organic feedstocks. In addition as described above, employment of this process step will provide additional nutrients as well as the recovered carbon.

### Detailed Description of the Preferred Embodiments

Referring now to the Figures in greater detail, there is illustrated therein a schematic representation of three embodiments of the system for carrying out the methodology of using high moisture renewable materials in the production of Syngas, although these should not be construed as limiting but merely as exemplary.

As an introduction, the process schematic consists of anaerobically treating the high moisture, renewable feedstock material in an anaerobic digestion (AD) process to produce a biogas primarily containing CH₄, CO₂ and smaller amounts of H₂S. The anaerobic digestion process can be accomplished using conventional CSTR, plug flow digesters, a dry (or solid state) anaerobic digester (generally with moisture levels of between 50% and 80%) or any number of other configurations such as two-stage and two phase digestion, a combination of a dry fermentation system with liquid recycle through a high rate anaerobic treatment reactor (such as an upflow anaerobic sludge bed (UASB) or higher rate variants such as the IC (Internal Circulation) or EGSB (Expanded Granule Sludge Bed), anaerobic fluidized bed reactor (AnFBR) or an anaerobic filter (AF)), as examples.

The high moisture content, renewable feedstock can be preconditioned to remove contaminants, reduce the particle size and/or thermally preprocessed to increase the rate and/or degree of degradation of the organics in the feedstock streams. The high moisture, renewable feedstock may also be pretreated to concentrate the organic content by removing water therefrom before entry into the anaerobic digester if desired. Mesophilic (˜35° C.), thermophilic (˜55° C.) or phased (use of both thermophilic and mesophilic steps in sequence) operating temperatures are both suitable for use in the anaerobic digestion system. The high moisture feedstock material stream can be processed alone or preferably in conjunction with processing of excess or waste biosolids generated from the Syngas fermentation process itself.

### Biogas Use

The biogas produced includes multiple components or fractions which are readily useable in the overall plant. Uses of the various fractions are described below.

Methane in the biogas may be used as a supplemental energy source in any device requiring a gaseous energy source such as steam boilers, thermal oxidizers, and burners or Steam Methane Reformers (SMR), as examples. The biogas may also be reformed to produce additional syngas. Although others have advocated that biogas can be reformed using any number of catalytic based reforming technologies, (such as Steam Methane Reformers [SMR] or Auto Thermal Reformers [ATR]) including Gunther U.S. Pat. No. 8,187,568B2 and Offerman U.S. Pat. No. 8,198,058 B2, this is expensive since the H₂S (and other reduced sulfur compounds in the biogas) and siloxanes present due to the partial degradation of silicone based compounds that are present in many high moisture feedstock's, require extensive and expensive pretreatment to protect the precious metal based catalysts. Therefore the preferred reforming technologies are non-catalytic partial oxidation (NC-PDX) or plasma arc-type gasification (either can use air or oxygen) since no pretreatment is needed and in fact the H₂S, useful as a sulfur source in the syngas fermentation, is, at least in part, preserved and present in the syngas produced and can be fed directly to the fermentation process where it can be used as a source of cell S. Other similar operational schemes to those listed above are possible by those skilled in the art.

If a NC-PDX or plasma gasifier is used to reform the biogas, the resultant syngas will have a relatively low e⁻/C ratio that usually does not exceed 4.0 and more often is less than 3.5, which indicates that not all the carbon oxides (CO₂ and CO) will be converted to the liquid products produced in the syngas fermentation system if just this syngas stream is used. This affords an opportunity to blend this gas with a second syngas that has a high e−/C that is usually at least 7.0 and more often at least 7.6 such as SMR reformed NG or certain waste industrial gases that tend to have high H₂ concentrations. The result is a high fraction of the carbon in both feedstocks (or combined syngas) can be converted to the soluble organic carbon compounds produced—an overall very effective carbon capture process. The electron to carbon ratio of the blended substrate gas will have an e⁻/C ratio in the range of about 5.4:1 to 6.7:1, preferably between about 5.6:1 to 6.5:1, and most preferably between about 5.8:1 to 6.3:1.

In another embodiment of the instant invention, an organic waste stream and excess biosolids (recovered, in part, from the Syngas fermentation) are co-digested in a conventional CSTR type anaerobic digestion (AD) system. The biogas produced in this case is not reformed but is forwarded to the primary Syngas fermentation reactor. It can be compressed and blended with the feed Syngas, or if fermenter tail gas recycle is employed, added to that stream on a low pressure side of a compressor or other such device used to recycle the gas. It can also be independently added at a point higher up in the fermenter where the need for CO₂ is the greatest. This allows the CO₂ in the biogas to be used to reduce the e⁻/C ratio into the preferred range in cases where the feed gas has relatively high e⁻/C ratio.

The CH₄ in the biogas is essentially inert in the Syngas fermentation process and passes with the tail gas which can be recovered and used for the energy value within the plant.

As mentioned, the reduced sulfur is used as an essential nutrient in anaerobic Syngas fermentation so the H₂S, if properly introduced into the fermentation process, can help offset the need for sulfur addition. The level of H₂S can be manipulated upward, if desired, by adding additional oxidized sulfur from sources, such as in the form of SO₄, to the primary anaerobic digestion system as well. Use of high oxidation state sulfur is described in U.S. patent application Ser. No. 13/546,703 filed Jul. 11, 2012 the contents of which are hereby incorporated in their entirety.

### Recovery of Nutrient Values

Through degradation processes occurring during anaerobic digestion, a significant fraction of the nitrogen (N) and phosphorus (P) organically bound in the organic materials being digested is transformed into recoverable inorganic, bioavailable forms useable in the fermentation process, primarily NH₄⁺/NH₃ and ortho PO₄. It is also possible to concurrently recover a significant fraction of the water needed for the fermentation with the recovered nutrients, if desired. The digestate stream may also contain other potentially valuable nutrients such as vitamins, amino acids, etc.

In one exemplary embodiment, the digestate stream is first sent to a liquid/solids separation unit, such as a centrifuge or screw press, and the centrate/pressate is then further processed to recover the N, P, and potentially, water, in a useable form. In one exemplary embodiment, this involves processing the centrate stream from a solids separation using a filtration process that includes a 1 to 10, preferably 1 to 5 kilodalton (kD) ultrafiltration (UF) membrane. The membrane system retains most of the larger Molecular Weight (MW) dissolved proteins, sugars and the like, whose build-up could adversely affect the syngas fermentation process, as a small volume retentate stream while providing a UF filtrate stream that contains most of the nutrient value of the digestate in a form that can be readily made suitable for addition to the fermenter.

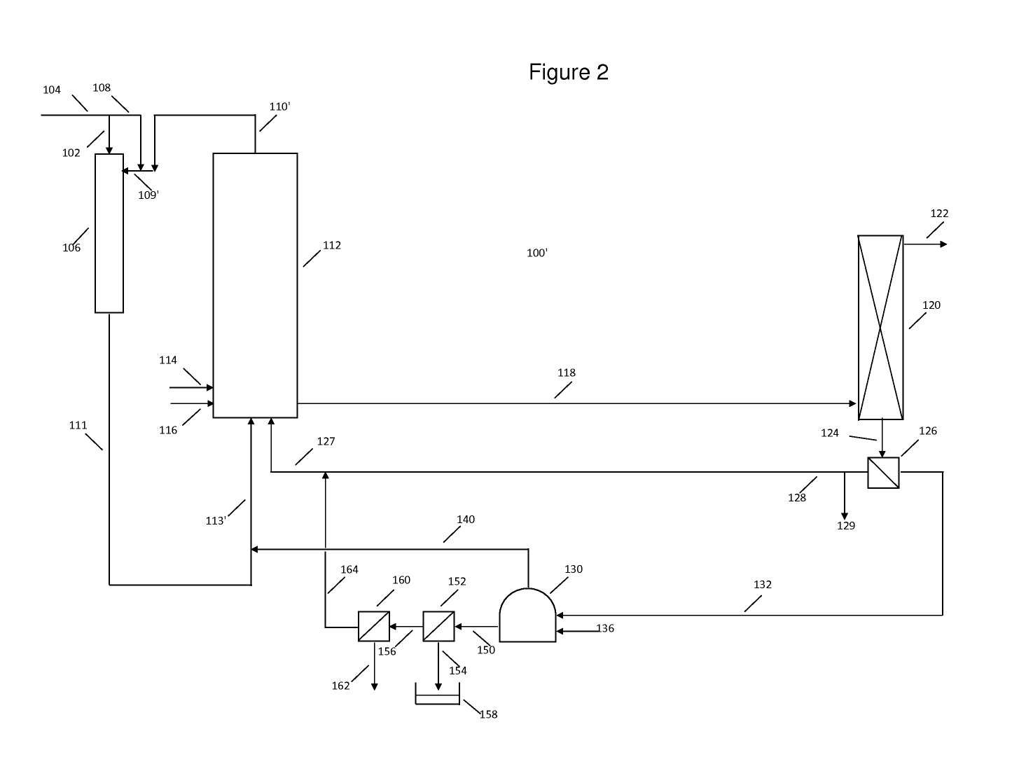

Additional treatment to remove remaining “contaminants” such as using granular activated carbon (GAC) adsorption and/or oxidation processes and/or thermal processing can be used to further clean and/or sterilize the UF filtrate prior to its use, if desired. What is shown in FIG. 2 is the centrate being processed in such a membrane system where the water, N and P are carried through as a permeate stream resulting in an N and P rich stream containing only very limited amounts of undesirable dissolved organics.

Other undesirable compounds such as large proteins, sugars, etc., captured in the retentate can be sent either to waste water treatment (WWT), or, if sufficiently concentrated, is sent to the AD system to recover the energy value. For example, this stream may also be sufficiently concentrated thorough evaporation to high total solids (TS) “syrup” which may be directly disposed of with the dewatered AD digestate solids, for example.

Other approaches to recovering these nutrients are possible, such as stripping/condensing or flashing ammonia from the AD centrate/pressate stream and/or precipitating the PO₄ in a form that can be subsequently recovered and then further processed so it can be used in the syngas fermentation, are also contemplated.

The systems illustrated would also allow recovery of approximately up to 80 to 90% of the water from the AD centrate/pressate as the permeate stream, which, in water shortage areas, could be extremely valuable. In addition, later portions of the text detail the amount of savings related to the recovery of the N, P, and S as well as the potential value of the generated CH₄ and CO₂.

### Recovery of Trace Metals

The dewatered AD digestate solids stream is generally between 18% and 25% total solids. This “cake” solids stream can be disposed of in a number of ways (land application, landfilling, etc.,) or it can be processed in a manner that allows recovery of the trace metals. To do this, the cake solids following dewatering are then dried to ˜90% to 92% TS and sent on to a gasifier. The syngas produced in the gasifier can be either added to the syngas “substrate” going to the fermentation system used to supply a significant fraction of the energy needed for drying the biosolids or used elsewhere in the plant for energy and/or heat. Waste heat from the overall syngas to chemicals process can be used to supply some or all of the heat required for drying which in many cases allows better use of the syngas produced.

It is possible to co-digest material that enhances the dewaterability of the AD digestate (due to additional fiber for example) and or increase the energy content of the digestate solids. This reduces the drying requirements and/or increases the net energy recovered during the gasification step.

The ash that is produced during gasification has a mass that is on the order of 3% to 5% of the Solids sent to gasification (on a dry weight basis) and can be landfilled, or land applied (perhaps qualify as biochar). This ash is, however, rich in phosphorus (P) and all the trace metals that are added to the fermentation process and taken up by the cells. In the syngas fermentation itself, citric acid is used as a chelant to prevent precipitation and ensure the bioavailability of certain required trace metals. Addition of citric acid (or other acceptable chelating agent) to the ash (that contains these metals) to achieve a pH of ˜3 to 4 is a sufficiently low pH to “leach” most of the metals and remaining P from the ash into the aqueous phase as a concentrated solution. This solution is then separated from the ash and cleaned as necessary for use back in the fermentation.

Most forms of fermentation zones (also called bioreactors) are suitable for use with the invention. The chief requirements of bioreactors include:
Axenicity;
Anaerobic conditions;
Suitable conditions for maintenance of temperature, pressure, and pH;
Sufficient quantities of substrates, and nutrients are supplied to the culture;
Optimum mass transfer performance to supply the gases to the fermentation medium
The end products of the fermentation can be readily recovered from the bacterial broth.

Types of fermentation apparatuses that are known to those of skill in the art, with or without additional modifications, or in other styles of fermentation equipment that are currently under development include but are not limited to: bubble column reactors; two stage bioreactors, trickle bed reactors; membrane bioreactors; packed bed reactors containing immobilized cells; stirred tank reactor; a column fermenter with immobilized or suspended cells; a continuous flow type bioreactor, a high pressure bioreactor, and a suspended cell reactor with cell recycle. These bioreactors may be used alone or in combination with multiple bioreactors of the same or different types in series or parallel flow. These apparatuses will be used to develop and maintain the microorganism cultures used to produce the Liquid Products of this invention. A new type of membrane bioreactor is a membrane supported bioreactor that supports microorganisms on the shell side of a membrane surrounded by a containment vessel and the fermentation broth passes through the lumen of the membrane. See U.S. Pat. No. 8,329,246.

In most cases the microorganisms will produce Liquid Products comprising alcohol. The Liquid Products produced in the processes of this invention will depend upon the microorganism used for the fermentation and the conditions of the fermentation. One or more microorganisms may be used in the fermentation broth to produce the sought alcohol. Bioconversions of CO and H₂/CO₂ to propanol, butanol, ethanol and other alcohols are well known. For example, in a recent book concise description of biochemical pathways and energetics of such bioconversions have been summarized by Das, A. and L. G. Ljungdahl, Electron Transport System in Acetogens and by Drake, H. L. and K. Kusel, Diverse Physiologic Potential of Acetogens, appearing respectively as Chapters 14 and 13 of Biochemistry and Physiology of Anaerobic Bacteria, L. G. Ljungdahl eds., Springer (2003). Any suitable microorganisms that have the ability to convert the Syngas components: CO, H₂, CO₂ individually or in combination with each other or with other components that are typically present in Syngas may be utilized. Suitable microorganisms and/or growth conditions may include those disclosed in U.S. patent application Ser. No. 11/441,392, filed May 25, 2006, entitled “Indirect Or Direct Fermentation of Biomass to Fuel Alcohol,” which discloses a biologically pure culture of the microorganism Clostridium carboxidivorans having all of the identifying characteristics of ATCC no. BAA-624; U.S. Pat. No. 7,704,723 entitled “Isolation and Characterization of Novel Clostridial Species,” which discloses a biologically pure culture of the microorganism Clostridium ragsdalei having all of the identifying characteristics of ATCC No. BAA-622; both of which are incorporated herein by reference in their entirety. Clostridium carboxidivorans may be used, for example, to ferment Syngas to ethanol and/or n-butanol. Clostridium ragsdalei may be used, for example, to ferment Syngas to ethanol.

Suitable microorganisms and growth conditions include the anaerobic bacteria Butyribacterium methylotrophicum, having the identifying characteristics of ATCC 33266 which can be adapted to CO and used and this will enable the production of n-butanol as well as butyric acid as taught in the references: “Evidence for Production of n-Butanol from Carbon Monoxide by Butyribacterium methylotrophicum,” Journal of Fermentation and Bioengineering, vol. 72, 1991, p. 58-60; “Production of butanol and ethanol from synthesis gas via fermentation,” FUEL, vol. 70, May 1991, p. 615-619. Other suitable microorganisms include: Clostridium Ljungdahlii, with strains having the identifying characteristics of ATCC 49587 (U.S. Pat. No. 5,173,429) and ATCC 55988 and 55989 (U.S. Pat. No. 6,136,577) that will enable the production of ethanol as well as acetic acid; Clostridium autoethanogemum sp. nov., an anaerobic bacterium that produces ethanol from carbon monoxide. Jamal Abrini, Henry Naveau, Edomond-Jacques Nyns, Arch Microbiol., 1994, 345-351; Archives of Microbiology 1994, 161: 345-351; and Clostridium Coskatii having the identifying characteristics of ATCC No. PTA-10522 U.S. Pat. No. 8,143,037, filed on Mar. 19, 2010. All of these references are incorporated herein in their entirety.

Suitable microorganisms for bioconversion of Syngas to Liquid Products generally live and grow under anaerobic conditions, meaning that dissolved oxygen is essentially absent from the fermentation liquid. Adjuvants to the fermentation broth may comprise buffering agents, trace metals, vitamins, salts etc. Adjustments in the fermentation broth may induce different conditions at different times such as growth and non-growth conditions which will affect the productivity of the microorganisms. U.S. Pat. No. 7,704,723, hereby incorporated by reference in its entirety, discloses the conditions and contents of suitable fermentation broth for bioconversion CO and H₂/CO₂ using anaerobic microorganisms.

Anaerobic fermentation conditions include a suitable temperature, usually between 25° and 60° C., frequently in the range of about 30° to 40° C. The conditions of fermentation, including the density of microorganisms, fermentation broth composition, and Syngas residence time, are preferably sufficient to achieve the sought conversion efficiency of hydrogen and carbon monoxide and will vary depending upon the design of the fermentation reactor and its operation. The pressure may be subatmospheric, atmospheric or super atmospheric, and is usually in the range of from about 90 to 1000 KPa absolute and in some instances higher pressures may be desirable for biofilm fermentation bioreactors. As most bioreactor designs, especially for commercial scale operations, provide for a significant height of fermentation broth for the fermentation, the pressure will vary within the fermentation bioreactor based upon the static head.

The fermentation conditions are preferably sufficient to convert at least about 85, preferably at least about 90, percent of the hydrogen in the substrate gas fed to the bioreactor assembly to alcohol. As stated above, a combination of bubble size and duration of contact with the aqueous fermentation broth are necessary to achieve these high conversions. For commercial operations, the fermentation operation preferably provides a total molar conversion of hydrogen and carbon monoxide in the substrate gas feed in the range of at least about 90, preferably at least about 95, mole percent. If required to provide adequate contact time between the gas bubbles and the aqueous fermentation broth, more than one bioreactor may be used in gas flow series in the bioreactor assembly. The use of sequential, deep tank bubble column bioreactors is disclosed in U.S. patent application Ser. No. 13/243,062, filed on Sep. 23, 2011, herein incorporated by reference in its entirety. Use of a single stage deep tank fermenter that can achieve this same conversion efficiency is disclosed in Ser. No. 13/243,159 filed on Sep. 23, 2011 and herein incorporated by reference in its entirety.

The rate of supply of the gas feed under steady state conditions to a fermentation bioreactor is preferably such that the rate of transfer of carbon monoxide and hydrogen to the liquid phase matches the rate that carbon monoxide and hydrogen are bioconverted. The rate at which carbon monoxide and hydrogen can be consumed will be affected by the nature of the microorganism, the concentration of the microorganism in the fermentation broth and the fermentation conditions. The rate of transfer of carbon monoxide and hydrogen to the fermentation broth is a parameter for operation, conditions affecting the rate of transfer such as interfacial surface area between the gas and liquid phases and driving forces are important.

Preferably the substrate gas (Syngas and any suitable recovered biogas) is introduced into the bioreactor in the form of microbubbles. Often the microbubbles have diameters in the range of 0.01 to 0.5, preferably 0.02 to 0.3 millimeter. Preferably the substrate gas is injected using a motive fluid. Variations in the motive liquid flow rate can be used to modulate the microbubble size and thus modulate the rate of transfer of carbon monoxide and hydrogen to the liquid phase. Moreover, the modulation provides microbubbles that provide a stable gas-in-liquid dispersion. The injectors may be jet mixers/aerators or slot injectors. Slot injectors are preferred, one form of which is disclosed in U.S. Pat. No. 4,162,970. The bubble size generated by the injectors will be influenced by, among other factors, the rate of liquid flow through the injector and the ratio of gas phase to liquid phase passing through the injector as well as characteristics of the fermentation broth itself including, but not limited to its static liquid depth. See also, U.S. patent application Ser. No. 13/243,062, filed on Sep. 23, 2011. In some instances the microbubbles which form a less dense gas-liquid dispersion and any motive fluid used to generate the microbubbles, can facilitate liquid mixing in a bioreactor.

Liquid products produced by the fermenter from the Syngas may be recovered by many suitable separation systems. Product recovery can consist of known equipment arrangements for, separation and recovery of liquid products from the fermentation liquid, removal of residual cell material and precipitated proteins and return of recovered fermentation liquid to the syngas fermentation and purging of waste streams and materials. Suitable equipment arrangements can include distillation columns, membrane systems, filters, centrifuges, cyclones, and other separation equipment. U.S. Pat. No. 8,211,679, herein incorporated by reference in its entirety, shows an arrangement for a product recovery bioreactor that recovers an ethanol product from a bioreactor.

The rate of withdrawal of the fermentation broth is typically sufficient to not exceed a desired maximum concentration of Liquid Product in the bioreactor since Liquid Products, as previously explained, can be inhibitory to the microorganisms. The maximum concentration is usually selected to assure that the microorganisms in the fermentation broth are not unduly adversely affected. The inhibitory concentration of an oxygenated organic compound depends upon the oxygenated organic compound (e.g., butanol is more inhibitory than ethanol) and the microorganism. However, at the same time higher concentrations of Liquid Product in the fermentation broth are preferred to facilitate and reduce costs of product separation, e.g., by distillation.

In preferred form the product recovery system includes a distillation column that receives the withdrawn fermentation broth from the bioreactor. The distillation column separates the dilute Liquid Product stream into an overhead vapor stream containing the Liquid Product and a product depleted bottoms stream. The dilute product stream enters near the top stage of the column. Separation requirements of the column will vary with the product concentration of the entering dilute product stream.

In case of a vacuum distillation column, the vacuum distillation column will normally operate at a pressure of about 200 torr to 500 torr. At higher product concentrations the vacuum column will normally provide at least 10 stages of separation. More typically the column will have about 15 stages of separation and will operate in the range of 300 to 400 torr. Ordinarily a vacuum column will provide an ethanol concentration of at least 30 wt % a more often at least 40 wt % in the overhead vapor stream. At low product concentrations, the vacuum conditions and stages of the separation will permit the column to operate with a relatively low maximum temperature of about 80 C.

In preferred form at least an aliquot portion of the withdrawn fermentation broth is continuously or intermittently passed to product recovery system such as distillation columns as described above. After alcohol recovery the bottoms pass in whole or in part to a solids separating unit operation to provide a solids depleted liquor and a solids rich stream. The portion passed to the solids separating unit operation is sufficient to provide the sought amount of solids removal and recovery, and if desired, the entire withdrawn fermentation broth can pass to the solids separation unit operation. The “clarified” broth can then return in whole or in part to the syngas fermentation. In some cases it is desirable to return the clarified broth into the syngas fermentation via a series of spray nozzles located above the liquid surface in the syngas fermenter. This helps provide foam suppression and scrubbing of the ethanol in the tail gas exiting the fermenters.

Any suitable solids separating unit operation may be used. Preferred unit operations are those using centrifugal forces such as disk stack centrifuges, decanter centrifuges, tubular and chamber bowl centrifuges, and imperforate basket centrifuges. The temperature of the fermentation broth passed to the solids separating unit operation may vary over a wide range. Generally the fermentation broth is provided without any significant cooling, e.g., in the range of about 30° C. to 40° C.

### Detailed Description of the Figures

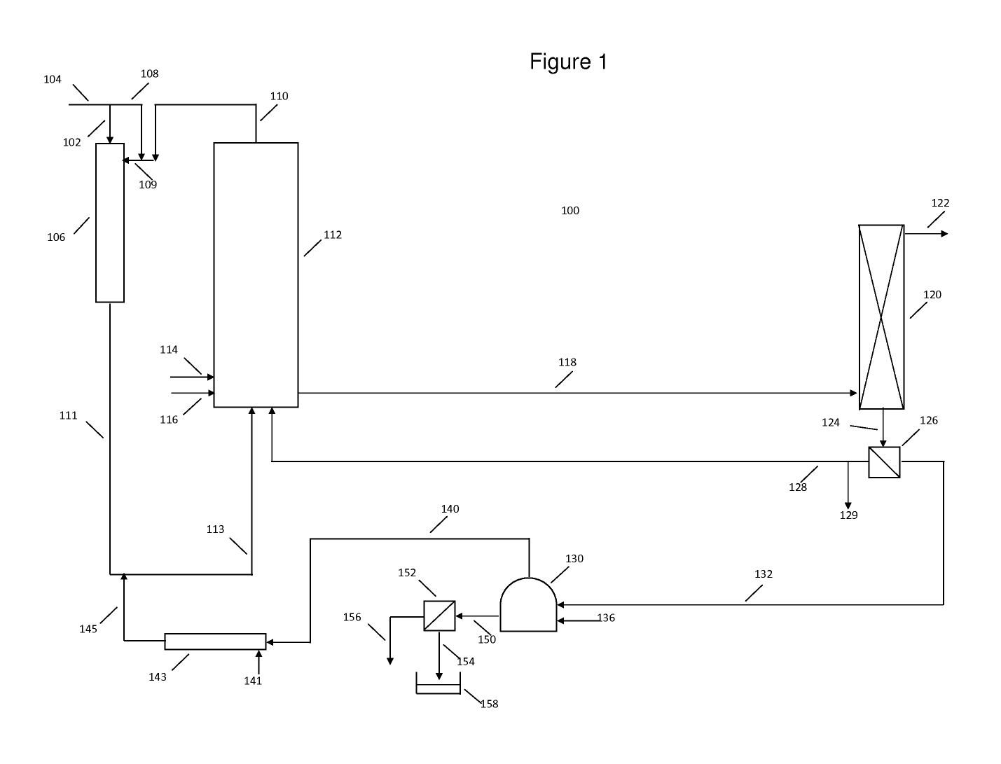

### FIG. 1

Shown in FIG. 1 is one embodiment of the system. NG from a suitable source (104) is delivered via a line 102 to reformer tubes of a steam methane reformer (SMR) 106 where the NG is converted to a Syngas with a high e⁻/C ratio. A portion of the NG from the source is also provided via a line 108 to fuel burners (not shown) in the reformer 106, together with tail gas from the fermenter via line 110, to supply all the energy needed for the burners via line 109. The Syngas formed in reformer 106 is fed via line 111 and 113 to fermenter 112 into which are also fed water via line 114 and nutrients via line 116 which are necessary for viability, reproduction and performance of the microorganisms within the fermenter 112, which microorganisms act on the Syngas to transform it into ethanol, in the disclosed embodiment. Any suitable microorganism can be utilized in the fermenter 112, as there are known a plurality of such which produce ethanol or others may be used to provide corresponding other desired products, as noted above.

A fermenter broth exiting fermenter 112 and comprising, in this instance, ethanol, water, and an amount of suspended biological solids, exits the fermenter via line 118 and is fed into a distillation tower 120. The ethanol is separated from water/solids contained in the product depleted broth and collected overhead from column 120 via line 122 for dehydration. The suspended solids and water, referred to as still bottoms, is fed via a line 124 to a suitable separator 126 for separation of solids from the liquid portion of the stream or still bottoms. The liquid portion of the stream, with solids removed therefrom, is referred to as clarified still bottoms, and is returned via line 128 to the fermenter 112 for further reprocessing. A slip stream of clarified bottoms can be sent to wastewater treatment either continuously or intermittently via line 129 to prevent the build-up of dissolved salts and/or biological metabolites to concentrations that can become inhibitory to the fermentation process. The solids containing stream separated from the clarified still bottoms, is carried via line 132 into any suitable anaerobic digester 130 where the solids are digested to produce biogas containing predominantly CH₄ and CO₂. Additional high moisture, renewable feed stock stream(s) can also be added to anaerobic digester 130 via line 136 to increase the amount of biogas produced and nutrients available for recovery and subsequent use back in the syngas fermentation system. Suitable sources for the high moisture, renewable feed stock stream may be provided in the form of food wastes, such as from cafes, cafeterias, grocery stores, hotels, homes, etc., in a preferred embodiment.

Digestion of the contents of the streams carried by lines 132 and 136 to anaerobic digester 132 will yield two streams, one of which is a CH₄ and CO₂ containing stream carried by line 140 to a non-catalytic partial oxidation reformer 143, which is concurrently fed a source of oxygen via line 141. The biogas and oxygen containing stream are reformed via non-catalytic partial oxidation reactor 143 into a syngas with a low e⁻/C ratio that is carried away via line 145. This syngas stream is then blended with the high e⁻/C ratio syngas in line 111 to produce a syngas with the optimal e⁻/C ratio to allow high conversion efficiencies of both the H₂/CO₂ and CO in the syngas, and sent to fermenter 112 in line 113. A liquid digestate stream also exits the anaerobic digester via line 150 and is carried to a solids separation unit 152 which separates and concentrates the digested solids which are removed via line 154 to storage tank 158 for transport off-site for various uses, such as land application for fertilization of soil, landfilling etc. The water stream from separation unit 152 is carried via a line 156 to a waste water treatment facility (not shown) for processing.

### FIG. 2

In the second embodiment shown in FIG. 2, the above description holds with the following modifications. In this operating scenario the biogas produced in digester 130 is carried via line 140 and blended with reformed NG gas stream 111 where the added CO₂ from the biogas is sufficient to generate a combined stream (line 113′) that has an optimal e⁻/C ratio to allow high conversion of the H₂/CO₂ and CO in fermenter 112. The tail gas that exits fermenter 112 via line 110′ has not only the unreacted H₂ and CO but all the CH₄ from the biogas in line 140. This stream is blended with sufficient NG from line 108 to produce a combined feed to the reformer hot box as line 109′ that has sufficient energy to operate the hot box of the reformer.

With regard to the water stream exiting the solids separation unit 152 via line 156, the water stream is taken by line 156 and fed through a UF filtration unit 160 that has a molecular weight cut off of between 1 to 5 KD. UF unit 160 retains most of the proteins and sugars as retentate stream 162 that is removed and sent to a waste water treatment facility. The cleaned, ultra-filtered water stream, or nutrient rich uncontaminated filtrate which is carried by line 164 can be blended with the flow in line 128 for delivery to the fermenter 112 as combined stream 127. The ultrafiltration system also removes contaminants such as bacteria which could adversely affect the microorganisms in fermenter 112. Note a pretreatment step prior to treatment of stream 156 in UF unit 160 may be needed in some cases (not shown).

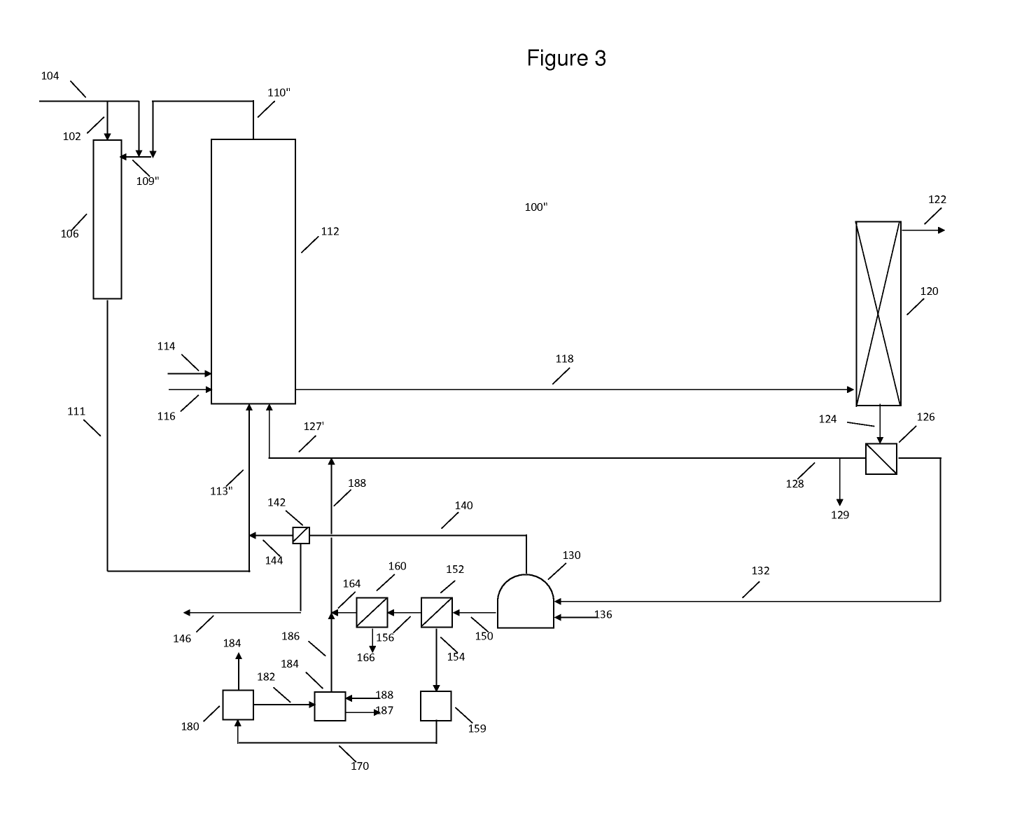

### FIG. 3

FIG. 3 has the same features as FIG. 2 with the following exceptions. The biogas in line 140 (from AD unit 130) is passed to a gas separation unit 142 that captures the CO₂ and H₂S which is then is taken in line 144 and blended with syngas feed in line 111 to generate a combined stream 113″ that has an e−/C ratio that allows high conversion efficiency of the H₂/CO₂ and CO in the syngas fed to fermenter 112. Any conventional CO₂ capture process, such as water scrubbing, solvent scrubbing (amines, selexol, etc.) or membrane separation processes can be used to capture the CO₂.

The reject stream 146 is primarily CH₄ that can be used for energy in the process. Use in the SMR would require removal of the siloxanes to a low level so is not a preferred use.

Additionally, in this figure the dewatered solids from digester 130 contained inline 154 are sent to dryer 159 where they are converted to stream 170 that has on the order of 8% to 10% moisture remaining. The solids in 170 are then fed to biosolids gasifier 180 that generates a syngas stream 190 that can be used for energy in the plant, used to dry the biosolids or even cleaned and used as feed to fermenter 112. The ash generated in gasifier 180 is carried away inline 182 to leach reactor 184. Citric acid is added via line 188 to 184 either continuously or batch-wise at sufficient amounts that a concentrated citric acid stream, rich in the trace metals and P, is leached from the ash and is recovered as stream 186. Stream 186 is blended with stream 164 forming stream 188 that is mixed with stream 128 and recycled back to fermenter 112 via line 127′. The remaining “leached” ash is removed for disposal via line 187.

As described above, the embodiments of the systems shown in FIGS. 1-3 provide a number of advantages, some of which are described above and others of which are inherent in the invention. Further, modifications may be proposed to the systems shown in FIGS. 1-3 and the other embodiments and aspects of the invention without departing from the teachings herein.

This calculated example involves the addition of source separated organics (SSO), particularly food wastes that have been treated to remove contaminants and macerated or pulped to generate a slurry suitable for co-digesting in an AD along with excess biosolids produced during the syngas fermentation. The resulting biogas stream is reformed using a non-catalytic partial oxidation reformer. This is in turn then blended with SMR reformed NG to produce a combined syngas with the preferred e−/C ratio to achieve a high conversion efficiency of both H2/CO2 and CO to soluble oxygenated products.

The SSO slurry is assumed to have a composition similar to that presented in Table 1, which is based on the characterization of a combined FW mixture from cafeterias, grocery markets and hotels (Zhang et al., 2007).

TABLE 1
Characterization of Food Wastes

| Parameter | Units | Concentration |
|---|---|---|
| TS | % | 30.9 |
| VS (volatile solids) | % | 26.3 |
| N | % dw | 3.16 |
| P | % dw | 0.52 |
| COD/VS | g/g | 1.55 |

This example assumes a nominal 12 million gallon/year (MGY) ethanol facility wherein 190 wet tons per day of raw SSO is processed, including removal of 5% of the total mass as contaminants, into a 10% TS slurry and co-digested with the excess biosolids from the fermentation process to produced biogas.

The composition of the NC-PDX reformed biogas, the SMR reformed NG streams and the blend of the two syngas's is shown in Table 2. The NC-PDX is fed with ˜650 scfm of CH4 along with 350 scfm of CO2. The SMR is fed with ˜1650 scfm of NG.

The PDX is used as the reformer for the biogas to reduce the amount of compression and eliminate any cleanup prior to reforming (no need for H₂S or siloxanes removal prior to the PDX—a considerable savings in capex and opex). This also preserves all of the carbon in the biogas and produces a syngas with a lower e⁻/C ratio that when blended with SMR reformed NG achieves the desired e⁻/C ratio for the fermentation process. A comparison of the composition of the two reformed gas streams and the blend that delivers the desired e⁻/C is presented in Table 2.

TABLE 2
Comparison of Different Syngas Compositions (mole fractions)

| | | | Blend of SMR and |
|---|---|---|---|
| Parameter | SMR-NG | POX-Biogas | POX |
| H2 | 0.752 | 0.451 | 0.691 |
| CO | 0.177 | 0.479 | 0.238 |
| CO2 | 0.062 | 0.070 | 0.063 |
| N2 | 0.004 | — | 0.003 |
| CH4 | 0.006 | — | 0.004 |
| e−/C Ratio | 7.77 | 3.39 | 6.17 |

Up to 80 gpm of water is recoverable with the UF membrane approach outlined previously. In such an approach there is an expected recovery of approximately 90% of the digestate stream as centrate from dewatering and that approximately 80% of the centrate stream can be recovered as UF permeate. The N, P and S requirements for the syngas fermentation process are shown in Table 3 along with the mass that can reasonably be recovered within this embodiment of the invention.

TABLE 3
Mass of nutrients that AD followed by
recovery of a centrate with a tight
(approximately 1 KD) UF membrane
can provide for Syngas in a
nominal 12 MGY Syngas to Ethanol Plant

| | | Needed | | | | |
|---|---|---|---|---|---|---|
| | | for | Available | Available | Available | Overall |
| | | Fermen- | from | from | from | Percent |
| Nutrient | Units | tation | Biosolids | SSO | Biogas | Recovered |
| Nitrogen | lb/d | 1,925 | 420 | 822 | | 95.8 |
| Phos- | lb/d | 162 | 52 | 85 | | 84.6 |
| phorus | | | | | | |
| Sulfur | lb/d | 130 | | | 60 | 46.2 |

Accordingly, the scope of the invention is only to be limited as necessitated by the accompanying claims.

The preamble to any claim in this invention is part of the entire claim and applies to interpreting the scope and coverage of each claim.

It is claimed:

1. A method for producing a liquid product via fermentation of a combination of biogas produced by the anaerobic digestion of high moisture organic feedstock (HMOF) and reformed natural gas (NG).

2. The method of claim 1 where the biogas is reformed using a non-catalytic partial oxidation reformer to produce a reformed biogas with a first e⁻/C ratio and the reformed natural gas has a second e⁻/C that exceeds the first e⁻/C and the portion of at least one of reformed biogas and the reformed natural gas is combined with at least a portion of the other gas in an amount to produce a combined syngas with a desired e⁻/C ratio.

3. The method of claim 2 wherein the first e⁻/C ratio is no greater than 4.0 and the second e⁻/C ratio is no less than 7.0.

4. The method of claim 1 where at least a portion of the HMOF is comprised of: excess biosolids produced in the syngas fermentation; organic fraction of municipal solid waste and biological waste sludge; source separated organics; green wastes from food supply or service operations including supermarkets and cafeterias; high strength food processing wastewaters including whey, whey permeate, and vegetable preparation; distillery waste waters including stillage and thin stillage; crop and agricultural residues; and, crops grown specifically as HMOF.

5. The method of claim 1 wherein the HMOF contains at least one of nitrogen and phosphorous in a form having limited bioavailability by microorganisms used in the fermentation and the anaerobic digestion of the HMOF converts at least a portion of the at least one of nitrogen or phosphorous to a form that has greater bioavailability relative to either of nitrogen or phosphorous present in the HMOF and at least one of the nitrogen or phosphorous is recovered from an aqueous phase that is separated from the digestate solids.

6. The method of claim 1 wherein the digestion of the HMOW produces a digester digestate stream from which an aqueous phase is removed to produce a liquid digestate stream and a solids digestate stream, the liquid digestate stream is treated to remove a majority of the suspended solids to produce a reduced solids digestate stream that passes through a tight UF membrane to produce a retentate stream and an ultra-filtered digestate stream that passes to the fermentation.

7. The method of claim 6 wherein at least a portion of the solids digestate is dewatered and dried, and the dried and dewatered digestate then passes to a gasifier to produce syngas that passes to the fermentation and an ash that contains trace metals.

8. The method of claim 7 wherein at least a portion of the ash is treated with citric acid to leach the metals into a solution and the trace metals and citric acid pass to the fermentation as a solution to provide both the trace metals and citric acid to the fermentation.

9. The method of claim 1 wherein the biogas is combined with the reformed natural gas after reforming of the biogas into syngas in a non-catalytic, partial oxidation reformer.

10. The method of claim 1 wherein a portion of the biogas passes to the burners of a reformer for producing the reformed natural gas.

11. The method of claim 10 wherein the biogas passes to a gas separation unit that separates methane from the biogas, at least a portion of the methane passes to the burners of the natural gas reformer and at least a portion of the remainder of the biogas passes to the fermentation in combination with the reformed natural gas.

12. The method of claim 1 wherein a portion of the biogas is reformed in a non-catalytic partial oxidation reformer into syngas that is at least in part combined with the reformed natural gas and a another portion of the biogas passes to the burners of a reformer that produces the reformed natural gas.

13. The method of claim 1 wherein the biogas passes into combination with the reformed natural gas without any reforming of the biogas wherein the biogas has a first e⁻/C ratio, the reformed natural gas has a second e⁻/C ratio that exceeds the first e⁻/C and the portion of at least one of reformed biogas and the reformed natural gas is combined with at least a portion of the other gas in an amount to produce a combined syngas with a desired e⁻/C ratio.

14. The method of claim 13 wherein the first e⁻/C ratio is no greater than 4.0 and the second e⁻/C ratio is no less than 7.0.

15. A method for producing an alcohol product via syngas fermentation of reformed natural gas (NG) from an NG reformer, a biogas produced by anaerobic digestion, and a tail gas that exits the fermentation, the process comprising:
a. digesting a high moisture organic feedstocks (HMOF) in a digester to produce biogas;
b. passing a portion of the reformed natural gas with the biogas to the fermentation to produce the alcohol product and the tail gas;
c. passing at least a portion of the tail gas as fuel to the burners of the NG reformer; and,
d. passing natural gas to the burners of the NG reformer to supply the remainder of the necessary fuel for the NG reformer.

16. The method of claim 15 where the biogas is reformed using a non-catalytic partial oxidation reformer to produce a syngas portion with a first e⁻/C ratio and an amount of reformed natural gas having a second e⁻/C that exceeds the first e⁻/C is combined with a the syngas portion in an amount to produce a combined syngas with a desired e⁻/C ratio

17. The method of claim 16 wherein the first e−/C ratio is no greater than 4.0, and the second e−/C ratio is no less than 7.0.

18. The method of claim 15 wherein the HMOF contains at least one of nitrogen and phosphorous in a form having limited bioavailability by microorganisms used in the fermentation and the anaerobic digestion of the HMOF converts at least a portion of the at least one of nitrogen or phosphorous to a form that has greater bioavailability relative to either of nitrogen or phosphorous present in the HMOF and at least one of the nitrogen or phosphorous is recovered from an aqueous phase that is separated from the digestate.

19. The method of claim 15 wherein the digestion of the HMOF produces a digester digestate stream from which an aqueous phase is removed to produce a liquid digestate stream and a solids digestate stream, the liquid digestate stream is treated to remove a majority of the suspended solids to produce a reduced solids digestate stream that passes through a tight UF membrane to produce a retentate stream and an ultra-filtered digestate stream that passes to the fermentation

20. The method of claim 19 wherein at least a portion of the solids digestate stream is dewatered to produce a dewatered digestate stream and dewatered digestate stream is dried and then passes to a gasifier for gasification to produce syngas that passes to the fermentation and to produce an ash that contains trace metals.

21. The method of claim 20 wherein at least a portion of the ash is treated with citric acid to leach the metals into a solution and the trace metals and citric acid pass to the fermentation as a solution to provide both the trace metals and citric acid in the syngas fermentation.

22. The method of claim 15 wherein the biogas passes to a gas separation unit that separates methane from the biogas, at least a portion of the methane passes to the burners of the natural gas reformer and at least a portion of the remainder of the biogas passes to the fermentation in combination with the reformed natural gas.

23. A method for producing an alcohol product via syngas fermentation of reformed natural gas (NG) from an NG reformer, a biogas produced by anaerobic digestion, and a tail gas that exits the fermentation, the process comprising:
a. digesting high moisture organic feedstocks (HMOF) and excess biosolids from the fermentation in a digester to produce the biogas and a digester digestate stream and reforming the biogas in a non-catalytic partial oxidation reformer to produce a reformed biogas;
b. passing at least a portion of the reformed natural gas with the reformed biogas to the fermentation to produce the alcohol product and the tail gas;
c. passing at least a portion of the tail gas as fuel to the burners of the NG reformer;
d. passing NG to the burners of the NG reformer to supply the remainder of the necessary fuel for the NG reformer;
e. separating water from the digester digestate stream to produce a liquid digestate stream and a solids digestate stream and the liquid digestate stream passes through a filtration system that includes a tight UF membrane to produce a retentate stream and an ultra-filtered water stream that passes to the fermentation;
f. at least a portion of the solids digestate stream is dewatered, dried and passes to a gasifier to produce syngas that passes to at least one of the fermentation, the burners of the NG reformer and an ash that contains trace metals; and,
g. at least a portion of the ash is treated with citric acid to leach the metals into a solution of trace metals and citric acid that pass to the fermentation as a solution to provide both the trace metals and citric acid to the fermentation.
